(19) United States
(12) Patent Application Publication
(10) Pub. No.: US 2021/0169726 A1
(43) Pub. Date: Jun. 10, 2021

(54) WALKING STATE DETERMINATION APPARATUS, WALKING ASSISTANCE APPARATUS, AND NON-TRANSITORY COMPUTER READABLE MEDIUM

(71) Applicant: Toyota Jidosha Kabushiki Kaisha, Toyota-shi (JP)

(72) Inventor: Uori Koike, Toyota-shi (JP)

(21) Appl. No.: 17/112,066

(22) Filed: Dec. 4, 2020

(30) Foreign Application Priority Data
Dec. 9, 2019 (JP) 2019-221879

(51) Int. Cl. A61H 1/02; A61B 5/11; A61B 5/00; A61H 3/00

(52) U.S. Cl. CPC A61H 1/024; A61B 5/1123; A63B 22/02; A61H 3/00; A63B 2220/20; A61B 5/6828

(57) ABSTRACT

To provide a walking state determination apparatus that can accurately detect a walking state of a person by performing simple calculation processing. The walking state determination apparatus determines the walking state of the person (for example, whether the state of the leg during the walking training is in a swing state or a stance state), and is suitable for determining the timing to assist a trainee. The walking state determination apparatus includes a distance measurement sensor attached to a prescribed part of a lower limb of the person and a calculation unit configured to determine the walking state of the person based on a distance from the distance measurement sensor to a floor surface detected by the distance measurement sensor.

### CROSS REFERENCE TO RELATED APPLICATIONS

This application is based upon and claims the benefit of priority from Japanese patent application No. 2019-221879, filed on Dec. 9, 2019 the disclosure of which is incorporated herein in its entirety by reference.

### BACKGROUND

The present disclosure relates to a walking state determination apparatus, a walking assistance apparatus, and a non-transitory computer readable medium.

A walking training system which is used when a trainee performs walking training is known. In the walking training system, since it is necessary to appropriately assist the trainee in walking in accordance with the walking state of the trainee, the walking state of the trainee who is under the training (for example, whether the state of the leg for which the walking training is performed is in a swing state or a stance state) needs to be determined. Patent Literature 1 (Japanese Unexamined Patent Application Publication No. 2013-123532) discloses a technique of detecting a plurality of discrimination periods (early/late swing phases and a stance phase) in the gait cycle using an acceleration sensor configured to detect acceleration of a thigh that rotates about a hip joint and an angular velocity sensor configured to detect an angular velocity of a lower-leg part that rotates about a knee joint in the walking motion. Patent Literature 1 further discloses that timings at which the thigh and the lower-leg part have electrical stimulation applied thereto are controlled based on the aforementioned detection result.

### SUMMARY

The technique disclosed in Patent Literature 1 has a problem that with the use of the acceleration sensor and the angular velocity senor, errors accumulate when the acceleration speed or the angular velocity information are integrated, and thus the walking state cannot be detected accurately. Further, there is also a problem that calculation processing (filtering processing) becomes complicated when the acceleration sensor or the angular velocity sensor is used.

The present disclosure has been made in view of the background mentioned above. An object of the present disclosure is to provide a walking state determination apparatus that can accurately detect a walking state of a person by performing simple calculation processing.

A first exemplary aspect is a walking state determination apparatus for determining a walking state of a person including:
a distance measurement sensor attached to a prescribed part of a lower limb of the person; and
a calculation unit configured to determine the walking state of the person based on a distance from the distance measurement sensor to a floor surface detected by the distance measurement sensor.

In the walking assistance system configured to assist the trainee in walking, it is necessary to determine the walking state of the trainee who is under the training in order to appropriately assist the trainee in walking in accordance with the trainee's walking state. In the walking state determination apparatus, a distance from a prescribed part of a lower limb of a person to which the distance measurement sensor is attached to the floor surface is detected by the distance measurement sensor while the person is walking. The distance from the distance measurement sensor attached to the prescribed part of the lower limb of the person to the floor surface detected by the distance measurement sensor changes while the person is walking. Therefore, the walking state of the person can be determined based on the distance to the floor surface detected by the distance measurement sensor. Further, it is possible to accurately determine the walking state of the person by performing, in the calculation unit, relatively simple calculation processing such as comparing time series data of the distance from the prescribed part of the lower limb of the person to the floor surface in the gait cycle acquired in advance with the detection value of the distance measurement sensor.

Further, the calculation unit may be configured to determine that the walking state of the person is at a timing at which the lower limb to which the distance measurement sensor is attached transits from a swing state to a stance state when the detection value of the distance measurement sensor that was exceeding a predetermined first threshold value falls below the first threshold value.

Further, the calculation unit may be configured to determine that the walking state of the person is at a timing at which the lower limb to which the distance measurement sensor is attached transits from a stance state to a swing state when the detection value of the distance measurement sensor that was equal to or lower than a predetermined second threshold value exceeds the second threshold value.

Further, the calculation unit may be configured to determine the walking state of the person according to a rate of change of the detection value of the distance measurement sensor.

In some embodiments, a position where the distance measurement sensor is attached is a calf region of the person. As a result of intensive research conducted by the present inventors, it was understood that when the distance measurement sensor is attached to a calf region of a person, the detection value of the distance from the calf region to which the distance measurement sensor is attached to the floor surface measured by the distance measurement sensor changed most conspicuously while the person was walking. Therefore, when the distance measurement sensor is attached to a calf region of a person, the walking state of the person can be distinguished particularly accurately.

A second exemplary aspect is a walking assistance apparatus configured to assist a motion of a knee joint of a trainee in accordance with a walking state of the trainee, the walking assistance apparatus including:
a damper configured to apply a resisting force in a flexing direction of the knee joint;
a control unit configured to control the resisting force of the damper; and
a walking state determination apparatus configured to determine the walking state of the trainee,
in which the walking state determination apparatus includes:
a distance measurement sensor attached to a prescribed part of a lower leg of the trainee; and
a calculation unit configured to determine the walking state of the trainee based on a distance from the distance measurement sensor to a floor surface measured by the distance measurement sensor, and
in which the control unit is configured to adjust the resisting force of the damper in accordance with the walking state of the trainee transmitted from the calculation unit.

With this configuration, a walking state of a person can be detected accurately by performing simple calculation processing whereby it is possible to appropriately assist a trainee in walking.

A third exemplary aspect is a non-transitory computer readable medium that stores a control program for a walking state determination apparatus for determining a walking state of a person, the control program configured to cause a computer of the walking state determination apparatus to execute the steps of:
detecting a distance from a distance measurement sensor attached to a prescribed part of a lower leg of the person to a floor surface by the distance measurement sensor; and
determining the walking state of the person based on the distance to the floor surface.

A walking state of a person can be accurately detected by performing simple calculation processing.

According to the present disclosure, it is possible to accurately detect the walking state of the person by performing simple calculation processing.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a perspective view illustrating a schematic configuration of a walking training system that employs a walking state determination apparatus according to an embodiment;

FIG. 2 is a front view of a walking assistance apparatus in the walking training system;

FIG. 3 is a perspective view illustrating a state in which the trainee is wearing the walking assistance apparatus;

FIG. 4 is a block diagram showing the walking training system;

FIG. 5 is a schematic diagram showing a configuration of the walking state determination apparatus according to an embodiment;

FIG. 6 is a schematic diagram for describing a method for determining a walking state of the trainee;

FIG. 7 is a flowchart showing a flow of processing for determining the walking state of the trainee in the walking state determination apparatus; and

FIG. 8 is a schematic diagram showing an example of a schematic configuration of a walking state determination apparatus according to a second modified embodiment which is of a type that is attached to a shoe.

### DESCRIPTION OF EMBODIMENTS

Hereinafter, the present disclosure will be described through embodiments of the present disclosure. However, the embodiments are not intended to limit the scope of the present disclosure according to the claims. For clarifying the explanation, the following description and the drawings are partially omitted and simplified where appropriate. The same symbols are assigned to the same elements in the drawings and duplicated explanations thereof are omitted where appropriate.

A walking state determination apparatus (system) according to the present embodiment is applied to a walking training system and determines a walking state of a person (a trainee). First, a configuration of a walking training system that employs the walking state determination apparatus according to the present embodiment will be described.

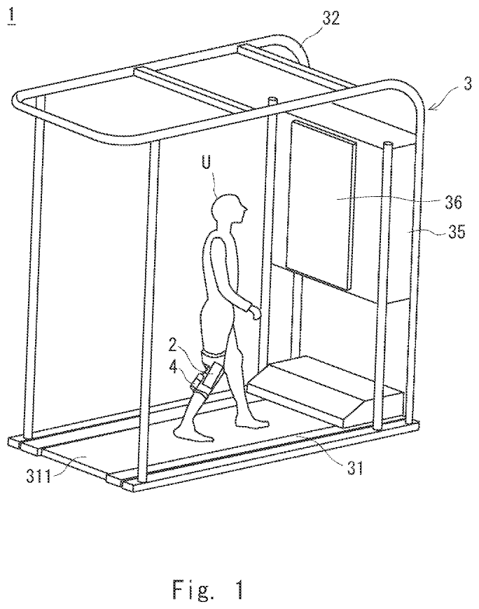

FIG. 1 is a schematic diagram for describing a configuration of a walking training system that employs a walking state determination apparatus according to a first embodiment. As shown in FIG. 1, a walking training system 1 includes a walking assistance apparatus 2 (system) worn on a leg part of a trainee U, a training apparatus 3 for performing a walking training of the trainee U, and a walking state determination apparatus 4.

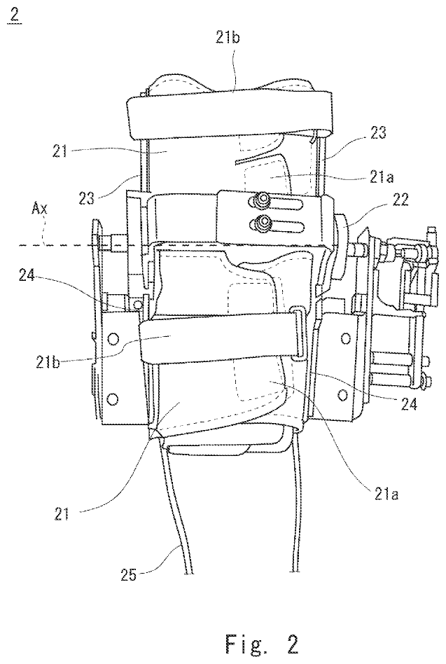

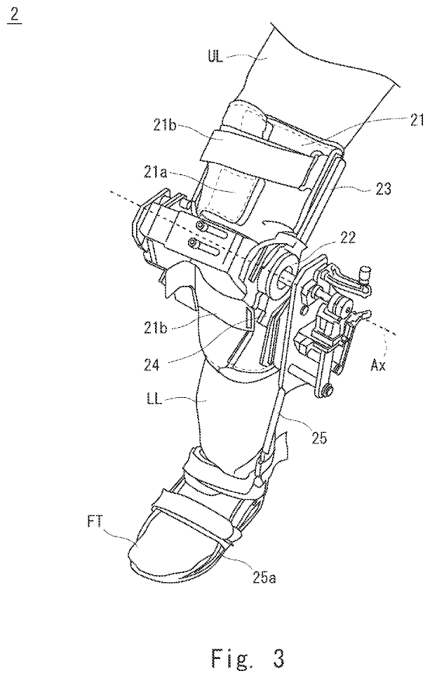

The walking assistance apparatus 2 is, for example, worn on an affected leg of the trainee U who performs the walking training and assists the trainee U in walking. The trainee U performs the walking training in a state in which the walking assistance apparatus 2 is worn on his/her knee joint. The walking assistance apparatus 2 applies a resisting force in a flexing direction of the knee joint. FIG. 2 is a front view of the walking assistance apparatus 2. FIG. 3 is a perspective view illustrating a state in which the trainee U is wearing the walking assistance apparatus 2. In FIG. 3, an example in which the trainee U is wearing the walking assistance apparatus 2 on his/her left leg is shown.

The walking assistance apparatus 2 includes a supporter 21, a damper 22, an upper leg frame 23, and a lower leg frame 24. A short lower limb gear 25 (partially omitted in FIG. 2) may be attached to a lower part of the walking assistance apparatus 2. The supporter 21 is formed of a stretchable material such as resin or fiber. The supporter 21 is worn on the knee joint and its surrounding, more specifically, from an upper leg UL across to a lower leg LL. Note that the upper leg UL corresponds to a part of the leg from the hip joint to the knee joint and the lower leg LL corresponds to a part of the leg from the knee joint to the ankle joint. The upper leg UL, the lower leg LL, and a sole FT are collectively referred to as the lower limb.

The supporter 21 includes a surface fastener 21a for wearing the walking assistance apparatus 2 on the knee joint. The trainee U winds the supporter 21 around the leg part and fixes it to the leg part with the surface fastener 21a. The surface fastener 21a is provided above and below the knee joint, specifically, to a front side of the upper leg UL and a front side of the lower leg LL. By using the surface fastener 21a, the trainee U can easily wear/take off the walking assistance apparatus 2. Further, it is possible to prevent the walking assistance apparatus 2 from being displaced from the knee joint of the trainee U. By employing the surface fastener 21a, the trainee U can adjust the degree of feeling of tightness. Further, a fixing band 21b may be provided in order to prevent the surface fastener 21a from being unfastened or the supporter 21 from being displaced.

The upper-leg frame 23 and the lower-leg frame 24 are attached to a side part of the supporter 21. The upper-leg frame 23 is arranged along the upper leg UL. The lower-leg frame 24 is arranged along the lower leg LL. The upper-leg frame 23 and the lower-leg frame 24 are connected to each other through the damper 22. The damper 22 is, for example, a rotary damper, and is located at a side part of the knee joint. Specifically, the damper 22 is positioned at the level of the knee joint so that a rotation axis Ax of the damper 22 roughly coincides with the axis of the knee joint. The upper-leg frame 23 and the lower-leg frame 24 configure a link mechanism that is rotatable about the rotation axis Ax of the damper 22.

The damper 22 applies a resisting force in the flexing direction of the knee joint. For example, the damper 22 reduces its speed of rotation in the flexing direction of the knee joint by utilizing the viscous drag of fluid such as oil. In some embodiments, the damper 22 is a unidirectional damper that applies the resisting force in only one direction. The damper 22 moves freely so as not to apply the resisting force in the extending direction of the knee joint. The damper 22 adjusts the resisting force by a control unit described later.

The short lower limb gear 25 is provided at a lower side of the supporter 21. As shown in FIG. 3, the short lower limb gear 25 is arranged along the lower leg LL of the trainee U. A sole frame 25a is provided at a lower end of the short lower limb gear 25. The sole frame 25a is fixed to the sole FT of the trainee U using a surface fastener or a fixing band.

Note that the configuration of the walking assistance apparatus 2 described above is merely an example and it is not to be limited thereto. It is possible to employ an arbitrary walking assistance apparatus that is worn on the leg part of the trainee U and assists the trainee in walking.

Referring again to FIG. 1, the training apparatus 3 includes a treadmill 31, a frame 32, and a control device 35. The treadmill 31 includes a rotatable ring-like belt conveyor 311 where the trainee U walks, and rotates the belt conveyor 311 at a set speed Vs. The trainee U stands on the belt conveyor 311 and walks thereon in accordance with the movement of the belt conveyor 311. A display unit 36 displays information, such as a training instruction to the trainee U, a training menu, and training information (walking speed, biological information etc.). For example, the display unit 36 may include a touch panel, in which case the trainee U can input various information through the display unit 36.

The control device 35 has a hardware configuration having a microcomputer at the center thereof, the microcomputer being configured of, for example, a CPU (Central Processing Unit) that performs operation processing, control processing etc., a ROM (Read Only Memory) that stores an operation program, a control program and the like executed by the CPU, a RAM (Random Access Memory) that stores various data, and an interface (I/F) that inputs/outputs a signal to/from the outside. The CPU, the ROM, the RAM, and the interface unit are connected with one another through, for example, a data bus.

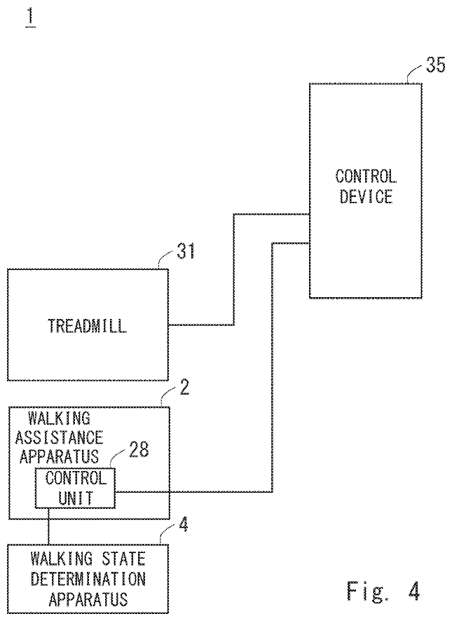

FIG. 4 is a block diagram of the walking training system 1. As shown in FIG. 4, the control device 35 is connected to the walking assistance apparatus 2 and the treadmill 31 via a wiring or the like. The control device 35 controls driving of the treadmill 31 based on information related to the walking state of the trainee U transmitted from the control unit 28 of the walking assistance apparatus 2. Further, the control device 35 causes the display unit 36 to display the training instruction to the trainee U, the training menu, the training information etc. based on the information related to the walking state of the trainee U transmitted from the control unit 28 of the walking assistance apparatus 2. The walking assistance apparatus 2 and the walking state determination apparatus 4 are connected to each other via a wiring or the like. The walking state of the trainee is transmitted from the walking state determination apparatus 4 to the control unit 28 of the walking assistance apparatus 2. The control unit 28 of the walking assistance apparatus 2 adjusts the resisting force of the damper in accordance with the walking state of the trainee transmitted from the walking state determination apparatus 4.

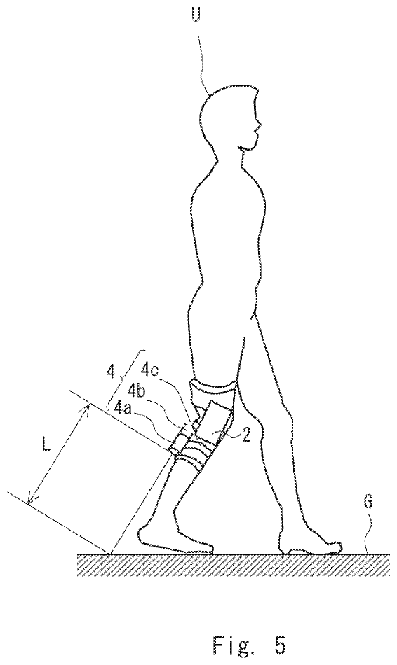

FIG. 5 is a schematic diagram showing a configuration of the walking state determination apparatus 4 according to this embodiment. FIG. 5 shows a state in which the walking state determination apparatus 4 is worn by the trainee U. Note that the walking state determination apparatus 4 according to this embodiment is incorporated in the walking training system 1, however for the sake of explanation, FIG. 5 shows a state in which only the walking state determination apparatus 4 is worn by the trainee U and other structural components in the walking training system 1 are omitted.

As shown in FIG. 5, the walking state determination apparatus 4 includes a distance measurement sensor 4a, a calculation unit 4b, and a fixing unit 4c. The distance measurement sensor 4a is attached to a prescribed part of the lower limb of the trainee U and measures a distance L from the prescribed part of the lower limb to a floor surface G. As the distance measurement sensor 4a, a general distance measurement sensor that measures a distance to a target object by irradiating light to the target object from a light source such as an LED or a laser diode and evaluating/calculating a reflection light from the target object which is received by a light receiving element can be used. As a measurement method of the distance measurement sensor, a general triangulation distance measurement method or a phase difference distance measurement method is generally used. The triangulation distance measurement method is a method in which a reflection light of a laser beam irradiated to a target object is read by a receiving device within the sensor and then a distance measurement method is performed based on the principle of triangulation. The phase difference distance measurement method is a method for measuring a distance by detecting “an optical phase difference” between a projected laser beam and its reflection light.

The fixing unit 4c is for fixing the distance measurement sensor 4a to a prescribed part of the trainee, and examples of which include a band such as a medical supporter. The position at which the distance measurement sensor 4a is attached is desirably a calf region of the lower limb of the trainee U. As a result of intensive research conducted by the present inventors, it was understood that when the distance measurement sensor 4a is attached to a calf region of a person, a detection value of the distance from the distance measurement sensor to the floor surface measured by the distance measurement sensor 4a changed most conspicuously while the person was walking. Therefore, when the distance measurement sensor 4a is attached to a calf region of a person, identification of the walking state of the person can be performed particularly accurately.

The calculation unit 4b determines the walking state of the trainee based on the distance L from the prescribed leg part to which the distance measurement sensor 4a is attached to the floor surface G detected by the distance measurement sensor 4a. The calculation unit 4b has a hardware configuration having a microcomputer at the center thereof, the microcomputer being configured of, for example, a CPU (Central Processing Unit) that performs operation processing, control processing etc., a ROM (Read Only Memory) that stores an operation program, a control program and the like executed by the CPU, a RAM (Random Access Memory) that stores various data, and an interface (I/F) that inputs/outputs a signal to/from the outside. The CPU, the ROM, the RAM, and the interface unit are connected with one another through, for example, a data bus.

Next, a method for determining the walking state of the trainee will be described. Note that in the following explanation, FIGS. 2, 3, and 5 are also referred to where appropriate.

In the walking training system 1, in order to appropriately assist the trainee in walking in accordance with the trainee's walking state, it is necessary to determine the trainee's walking state during the training. In the walking motion, while the trainee's leg on which the walking assistance apparatus 2 is worn (here, the right leg) is in the stance state, the force applied on the leg is relatively large whereas while the trainee's leg on which the walking assistance apparatus is worn is in the swing state, the force applied on the leg is relatively small. Therefore, the walking assistance apparatus 2 needs to assist the trainee in walking at least while the trainee's leg on which the walking assistance apparatus 2 is worn is in the stance state by making the resistance value of the damper of the walking assistance apparatus 2 relatively large. That is, it is necessary to detect a timing at which the leg on which the walking assistance apparatus 2 is worn transits from the swing state to the stance state (a first timing), and to make the resistance value of the damper of the walking assistance apparatus 2 relatively large.

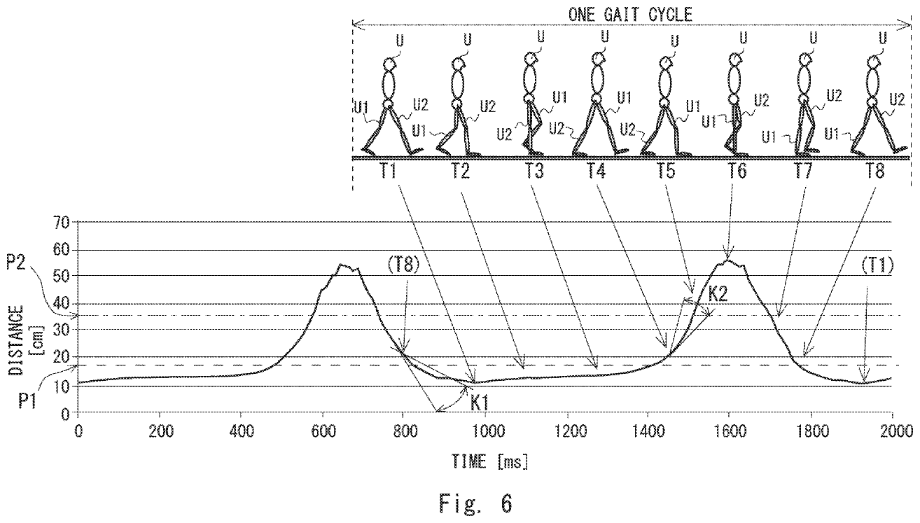

FIG. 6 is a schematic diagram that describes a method for determining a walking state of the trainee. A graph shown in FIG. 6 shows an example of changes in a distance from a calf region of a trainee's right leg on which the distance measurement sensor 4a is worn to the floor surface while the trainee is walking. Here, the vertical axis indicates a distance [cm] detected by the distance measurement sensor 4a from the calf region of the trainee's right leg on which the distance measurement sensor 4a is worn to the floor surface, and the horizontal axis indicates a time [ms].

Further, stratification of a gait cycle according to the “Rancho Los Amigos method” commonly used in clinical walking analysis is shown in correspondence with the graph shown in FIG. 6. Here, a normal-leg of the trainee U is indicated by U1 and an affected-leg of the trainee U on which the walking assistance apparatus 2 and the distance measurement sensor 4a are worn is indicated by U2. The gait cycle is stratified into eight strata of T1 to T8. T1 indicates initial contact, T2 indicates loading response, T3 indicates mid-stance, and T4 indicates terminal stance. Further, T5 indicates pre-swing, T6 indicates initial swing, T7 indicates mid-swing, and T8 indicates terminal stance.

In the graph shown in FIG. 6, the distance from the distance measurement sensor 4a to the floor surface detected by the distance measurement sensor 4a becomes smaller than a first threshold value P1 shown by the dotted lines while transition is made from the terminal stance T8 to the initial contact T1. Therefore, the calculation unit 4b of the walking state determination device 4 determines that it is a timing of transition from the swing state to the stance state (a first timing) when the detection value of the distance measurement sensor 4a that was exceeding a predetermined first threshold value P1 falls below the first threshold value P1. The first threshold value P1 is determined from the time series data of the detection value of the distance measurement sensor 4a acquired in advance while the trainee was walking. As described above, by determining the first timing by the calculation unit 4b, it is possible to accurately detect the timing of transition from the swing state to the stance state; that is, the timing at which the resistance value of the damper of the walking assistance apparatus 2 is made relatively large, by performing simple calculation processing.

As described above, in the walking motion, while the trainee's leg on which the walking assistance apparatus 2 is worn is in the swing state, the force applied on the leg is relatively small. Therefore, while the trainee's leg on which the walking assistance apparatus 2 is worn is in the swing state, the resistance value of the damper of the walking assistance apparatus 2 is made relatively small. In order to make the resistance value of the damper of the walking assistance apparatus 2 relatively small while the trainee's leg is in the swing state, a timing of transition from the swing state to the stance state (a second timing) needs to be detected.

In the graph shown in FIG. 6, while transition is made from the terminal stance T4 to the pre-swing T5, the distance from the distance measurement sensor 4a to the floor surface detected by the distance measurement sensor 4a exceeds a second threshold value P2 shown by the dashed-dotted lines. Therefore, the calculation unit 4b of the walking state determination device 4 determines that it is the timing of transition from the stance state to the swing state (the second timing) when the detection value of the distance measurement sensor 4a that was equal to or lower than the predetermined second threshold value P2 exceeds the second threshold value P2. The second threshold value P2 is determined from the time series data of the detection value of the distance measurement sensor 4a acquired in advance when the trainee was walking. As described above, by determining the second timing by the calculation unit 4b, it is possible to accurately detect the timing of transition from the stance state to the swing state, that is, the timing at which the resistance value of the damper of the walking assistance apparatus 2 is made relatively large, by performing simple calculation processing.

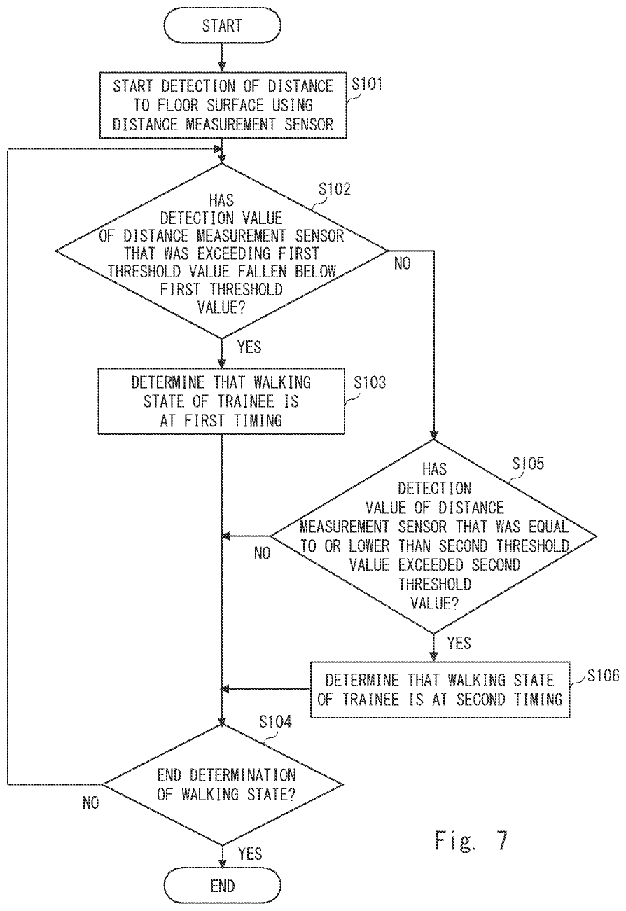

FIG. 7 is a flowchart showing a flow of processing for determining the walking state of the trainee performed in the calculation unit 4b of the walking state determination apparatus 4. As shown in FIG. 7, first, the distance measurement sensor 4a starts detection of the distance from the distance measurement sensor 4a to the floor surface (Step S101). Then, the distance measurement sensor 4a determines whether or not the detection value of the distance measurement sensor 4a that was exceeding the predetermined first threshold value falls below the first threshold value (Step S102). When it is determined in Step S102 that the detection value of the distance measurement sensor 4a that was exceeding the predetermined first threshold value falls below the first threshold value, it is determined that the timing is the first timing at which the walking state of the trainee transits from the swing state to the stance state (Step S103). Next, whether or not to end the determination of the walking state is determined (Step S104). When it is determined in Step S104 that the determination of the walking state is to be ended, the processing ends. When it is not determined in Step S104 that the determination of the walking state is to be ended, the processing returns to Step S102.

When it is not determined in Step S102 that the detection value of the distance measurement sensor that was exceeding the predetermined first threshold value falls below the first threshold value, whether or not the detection value of the distance measurement sensor 4a that was equal to or lower than the predetermined second threshold value exceeds the second threshold value is determined (Step S105). When it is determined in Step S105 that the detection value of the distance measurement sensor 4a that was equal to or lower than the predetermined second threshold value exceeds the second threshold value, it is determined that the timing is the second timing at which the walking state of the trainee transits from the stance state to the swing state (Step S106), and the processing proceeds to Step S104. When it is not determined in Step S105 that the detection value of the distance detection sensor 4a that was equal to or lower than the predetermined second threshold value exceeds the second threshold value, the processing proceeds to Step S104.

The control unit 28 of the walking assistance apparatus 2 shown in FIG. 4 makes the resistance value of the damper of the walking assistance apparatus 2 relatively large at the first timing detected by the walking state determination apparatus 4. By this configuration, the walking motion of the trainee is less assisted while the trainee's leg on which the walking assistance apparatus 2 is worn is in the stance state. Further, the control unit 28 makes the resistance value of the damper of the walking assistance apparatus 2 relatively small at the second timing detected by the walking state determination apparatus 4. By this configuration, the walking motion of the trainee is less assisted while the trainee's leg on which the walking assistance apparatus 2 is worn is in the swing state. By this configuration, it is possible to appropriately assist walking of the trainee using the walking assistance apparatus 2.

Making the resistance value of the damper of the walking assistance apparatus 2 relatively small includes the case where the resistance value of the damper is brought to zero (0). In this case, on/off control of turning-on the damper of the walking assistance apparatus 2 at the timing of transition from the swing state to the stance state and turning-off the damper of the walking assistance apparatus 2 at the timing of transition from the stance state to the swing state is performed in the control unit 28.

As described above, in the walking assistance apparatus 2 that assists the trainee in walking, it is necessary to determine the trainee's walking state during the training in order to appropriately assist the trainee in walking in accordance with the trainee's walking state. In the walking state determination apparatus 4, a distance from the distance measurement sensor 4a attached to a prescribed part of a lower limb of a person to the floor surface is detected by the distance measurement sensor 4a while the person is walking. The distance from the prescribed part of the lower limb of the person on which the distance measurement sensor 4a is attached to the floor surface detected by the distance measurement sensor 4a changes while the person is walking. Therefore, the walking state of the person can be determined based on the distance from the distance measurement sensor 4a to the floor surface detected by the distance measurement sensor 4a. Further, it is possible to accurately determine the walking state of the person by performing a relatively simple calculation processing such as comparing time series data of the distance from the prescribed part of the lower limb of the person to the floor surface in the gait cycle acquired in advance with the detection value of the distance measurement sensor 4a in the calculation unit 4b.

### MODIFIED EXAMPLE 1

Determination of the walking state in the calculation unit 4b may be performed on the basis of a rate of change of the detection value of the distance measurement sensor 4a instead of on the basis of the predetermined threshold value. Specifically, the calculation unit 4b determines the first timing which is the timing of transition from the swing state to the stance state (the timing of transition from T8 to T1) based on whether or not the rate of change of the detection value of the distance measurement sensor 4a, that is, the inclination of the graph shown in FIG. 6, is within a first inclination range K1. In the similar manner, the calculation unit 4b determines the second timing which is the timing of transition from the stance state to the swing state (the timing of transition from T4 to T5) based on whether or not the inclination of the graph is within a second inclination range K2. Note that the first and the second inclination ranges are determined from the data of temporal change of the detection value of the distance measurement sensor 4a acquired in advance while the trainee was walking. By this configuration, it is possible to accurately detect the walking state by performing simple calculation processing.

### MODIFIED EXAMPLE 2

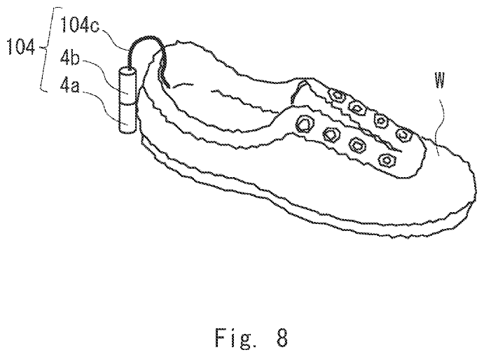

As described above, the distance measurement sensor of the walking state determination apparatus may be attached to either one of the trainee's lower limbs on which the walking assistance apparatus is worn. Therefore, the distance measurement sensor of the walking state determination apparatus may be attached to a shoe worn on either one of the lower limbs on which the walking assistance apparatus is worn. FIG. 8 is a schematic diagram showing an example of a schematic configuration of a walking state determination apparatus 104 according to a second modified example which is of a type that is attached to a shoe W. The walking state determination apparatus 104 includes the distance measurement sensor 4a, the calculation unit 4b, and the fixing unit 104c. As shown in FIG. 8, the fixing unit 104c of the walking state determination apparatus 104 is a clip with which the walking state determination apparatus 104 can be attached/detached to/from the heel of the shoe W. By configuring the walking state determination apparatus 104 as described above, the walking state determination apparatus 104 can be easily attached/detached.

Note that the present disclosure is not limited to the above-described embodiments, and can be appropriately changed without departing from the spirit of the present disclosure.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A walking state determination system for determining a walking state of a person comprising:
a distance measurement sensor attached to a prescribed part of a lower limb of the person; and
a calculation unit configured to determine the walking state of the person based on a distance from the distance measurement sensor to a floor surface detected by the distance measurement sensor.

2. The walking state determination system according to claim 1, wherein the calculation unit is configured to determine that the walking state of the person is at a timing at which the lower limb to which the distance measurement sensor is attached transits from a swing state to a stance state when a detection value of the distance measurement sensor that was exceeding a predetermined first threshold value falls below the predetermined first threshold value.

3. The walking state determination system according to claim 2, wherein the calculation unit is configured to determine that the walking state of the person is at a timing at which the lower limb to which the distance measurement sensor is attached transits from the stance state to a swing state when the detection value of the distance measurement sensor that was equal to or lower than a predetermined second threshold value exceeds the predetermined second threshold value.

4. The walking state determination system according to claim 2, wherein the calculation unit is configured to determine the walking state of the person based on a rate of change of the detection value of the distance measurement sensor.

5. The walking state determination system according to claim 1, wherein a position where the distance measurement sensor is attached is a calf region of the person.

6. A walking assistance system configured to assist a motion of a knee joint of a trainee in accordance with a walking state of the trainee, the walking assistance system comprising:
a damper configured to apply a resisting force in a flexing direction of the knee joint;
a control unit configured to control the resisting force of the damper; and
a walking state determination system configured to determine the walking state of the trainee,
wherein the walking state determination system comprises:
a distance measurement sensor attached to a prescribed part of a lower leg of the trainee; and
a calculation unit configured to determine the walking state of the trainee based on a distance from the distance measurement sensor to a floor surface measured by the distance measurement sensor, and
wherein the control unit is configured to adjust the resisting force of the damper in accordance with the walking state of the trainee transmitted from the calculation unit.

7. A non-transitory computer readable medium configured to store a control program for a walking state determination system for determining a walking state of a person, the program configured to cause a computer of the walking state determination system to execute the steps of:
detecting a distance from a distance measurement sensor attached to a prescribed part of a lower leg of the person to a floor surface by the distance measurement sensor; and
determining the walking state of the person based on the distance from the distance measurement sensor to the floor surface.

8. A method for a walking state determination system for determining a walking state of a person, the method configured to cause a computer of the walking state determination system to execute the steps of:
detecting a distance from a distance measurement sensor attached to a prescribed part of a lower leg of the person to a floor surface by the distance measurement sensor; and
determining the walking state of the person based on the distance from the distance measurement sensor to the floor surface.
